(19) United States
(12) Patent Application Publication
(10) Pub. No.: US 2005/0049196 A1
(43) Pub. Date: Mar. 3, 2005

(54) Indirect delivery of growth factors into the central nervous system

(76) Inventors: Michael Hutchinson, New York, NY (US); John Gianutsos, New York, NY (US)

Correspondence Address: Kenneth I. Kohn;Kohn & Associates, PLLC, Suite 410, 30500 Northwestern Hwy., Farmington Hills, MI, 48334, US

(21) Appl. No.: 10/927,301

(22) Filed: Aug. 26, 2004

Related U.S. Application Data
(60) Provisional application No. 60/499,232, filed on Aug. 29, 2003.

(51) Int. Cl. A61K 38/18

(52) U.S. Cl. 514/12

(57) ABSTRACT

The present invention provides a method of delivering a treatment to the central nervous system, including the steps of administering a therapeutic intramuscularly into muscles innervated by nerves chosen from the group consisting of cranial nerves and spinal nerves, and transporting the therapeutic peripherally through nerves into the CNS and to the site of treatment. The present invention provides a method of treating a neural disorder or a part of the body by administering an effective amount of a compound affecting neural cells into muscles innervated by cranial and/or spinal nerves, and affecting neural cells. The present invention also provides a method of treating a spinal cord injury by administering an effective amount of a compound affecting neural cells into musculature directly innervated by the spinal cord, and affecting neural cells. The present invention further provides a method of treating Parkinson's disease by administering an effective amount of a compound affecting neural cells into musculature innervated by the motor trigeminal nerve, and affecting neural cells involved in Parkinson's disease. Finally, the present invention also provides a method of treating amyotrophic lateral sclerosis by administering an effective amount of a compound affecting neural cells into musculature innervated by cranial and/or spinal nerves, and affecting neural cells involved in amyotrophic lateral sclerosis.

### CROSS REFERENCE TO RELATED APPLICATIONS

This application claims the benefit of priority under 35 U.S.C. Section 119(e) of U.S. Patent Application No. 60/499,232, filed Aug. 29, 2003, which is incorporated herein by reference.

### TECHNICAL FIELD

The present invention relates to the field of medical treatment and to the field of drug delivery and in vivo transport. More specifically, the present invention is directed towards medical treatment of disorders of the central nervous system.

### BACKGROUND

Numerous neurological disorders exist and are incurable. Whether the neurological disorder is a disease of the central nervous system (CNS) and/or peripheral nervous system (e.g., Parkinson's disease, amyotrophic lateral sclerosis (Lou Gehrig's Disease), etc.) or nerve injury or trauma associated with spinal cord injury, numerous treatments are being studied and tested. One particular disease of interest is Parkinson's disease.

Parkinson's disease is a disturbance of voluntary movement in which muscles become stiff and sluggish. Symptoms of the disease include difficult and uncontrollable rhythmic twitching of groups of muscles that produces shaking or tremors. Currently, the disease is caused by degeneration of pre-synaptic dopaminergic neurons in the brain and specifically in the brain stem. As a result of the degeneration, an inadequate release of the chemical transmitter dopamine occurs during neuronal activity.

Amyotrophic lateral sclerosis (ALS), also called Lou Gehrig's disease, is a progressive, fatal neurological disease. ALS occurs when specific nerve cells in the brain and spinal cord that control voluntary movement gradually degenerate and causes the muscles under their control to weaken and waste away, leading to paralysis. Currently there is no cure for ALS; nor is there a proven therapy that will prevent or reverse the course of the disorder.

Although Parkinson's disease can affect tissue throughout the brain, typically most of the damage is found throughout the brainstem. In particular, the substantia nigra (hereinafter, “SN”) is severely affected in every case. Connections of the motor trigeminal nerve (hereinafter, “MTN”) to the SN have been demonstrated from the SN (See, Marchand, et al.). In addition, extensive brainstem connectivity has been demonstrated (See, for example, Fay, RA et al. and Marchand, et al.).

Currently, Parkinson's disease is treated with several different compounds and combinations. Levodopa (L-dopa), which is converted into dopamine in the brain, is often given to restore muscle control. Perindopril, an ACE inhibitor that crosses the blood-brain barrier, is used to improve patients' motor responses to L-dopa. Carbidopa is administered with L-dopa in order to delay the conversion of L-dopa to dopamine until it reaches the brain, and it also lessens the side effects of L-dopa. Other drugs used in Parkinson's disease treatment include dopamine mimickers Mirapex (pramipexole dihydrochloride) and Requip (ropinirole hydrochloride), and Tasmar (tolcapone), a COMT inhibitor that blocks a key enzyme responsible for breaking down levodopa before it reaches the brain.

There are also other surgical treatments such as Activa® Tremor Control System (Medtronic), which consists of a wire surgically implanted deep within the brain and connected to a pulse generator, similar to a cardiac pacemaker, implanted near the collarbone. Whenever a tremor begins, patients can activate the device by passing a hand-held magnet over the generator. Pallidotomy can also be performed, wherein a pearl-sized heat lesion is made using refined stereotactic techniques to correct abnormally discharging nerve cells located in the globus pallidus internus.

Although there are numerous compounds and/or compositions believed to treat neurological disorders such as Parkinson's disease or nerve damage from spinal cord injury, studies are still currently being pursued. Recent advances in the treatment of Parkinson's disease and spinal cord injuries, however, have suggested that glial-derived neurotrophic factor (hereinafter, “GDNF”) can cause regression of symptoms associated with Parkinson's disease and spinal cord injuries. Specifically, studies of dopaminergic function in these patients suggest that there can be regression of the disease process itself (Gill, S G et al.). Injecting GDNF into the spinal cord of rats has shown to increase neuronal fibers with calcitonin gene-related peptide, neurofilament, and growth-associated protein 43 immunoreactivity in injured spinal tissue and also to cause 50% cell survival in contused spinal cord tissues (Cheng, H. et at.). Unfortunately, GDNF does not cross the blood-brain barrier and so cannot be given in a conventional way such as oral, intravenous, or subcutaneous administration. GDNF is injected into the brain because the protein must be injected right into the ventricular system in which the brain floats. For example, surgeons can implant a small bubble, which acts as a reservoir, just under the patient's scalp. The bubble has a small straw at the end that leads directly to the ventricles. Once a month, patients come into the hospital and doctors inject GDNP into the reservoir. However, there are no current methods of administering GDNP without injecting it into the brain.

Axonal transport is one of the body's own transport mechanisms. Axonal transport of horseradish peroxidase has been shown in rats when injections were made in dorsal, lateral, and medial areas of the periaqueductal gray in the brain (Marchand, J. E. et al.). Retrogradely-labeled cells were found to have traveled to various parts of the brain such as the lateral reticular nucleus of the medulla, nucleus raphe magnus, nucleus reticularis pontis caudalis, locus ceruleus, dorsal and ventral parabrachial nuclei, substantia nigra, and the lateral hypothalamus. Axonal transport has also been used to deliver painkilling drugs to specifically targeted nerves.

Intranasal delivery has been used as a noninvasive method of bypassing the blood-brain barrier in order to deliver drugs to the central nervous system. Delivery is possible because of the connection between the olfactory and trigeminal nerves, and the brain and the external environment. Neurotrophic factors such as NGF, IGF-1, FGF and ADNF12 have been delivered intranasally to the CNS. While intranasal delivery is promising, there are also some concerns with higher molecular weight drugs, degradation of drugs in the nasal mucosa, irritation to the mucosa, and interference with drug delivery from nasal congestion.

Accordingly, there is a need for a method of treating diseases that affect the CNS. Specifically, there is a need for a method of administering and transporting compounds that otherwise cannot cross the blood-brain barrier.

### SUMMARY OF THE INVENTION

The present invention provides a method of delivering a treatment to the central nervous system by administering a therapeutic intramuscularly into muscles innervated by nerves chosen from the group consisting of cranial nerves and spinal nerves, and transporting the therapeutic peripherally through nerves into the CNS and past the blood-brain barrier.

The present invention provides a method of treating a neural disorder by administering an effective amount of a therapeutic into muscles innervated by cranial and/or spinal nerves, wherein the therapeutic affects neural cells, transporting the therapeutic peripherally through nerves into the CNS and past the blood-brain barrier, and affecting neural cells.

The present invention also provides a method of treating a spinal cord injury by administering an effective amount of a therapeutic into musculature directly innervated by the spinal cord, wherein the therapeutic affects neural cells, transporting the therapeutic peripherally through nerves into the CNS and past the blood-brain barrier, and affecting neural cells.

The present invention also provides a method of treating Parkinson's disease by administering an effective amount of a therapeutic into musculature innervated by the motor trigeminal nerve, wherein the therapeutic affects neural cells, transporting the therapeutic peripherally through nerves into the CNS and past the blood-brain barrier, and affecting neural cells involved in Parkinson's disease.

The present invention also provides a method of treating amyotrophic lateral sclerosis by administering an effective amount of a therapeutic into musculature innervated by cranial and/or spinal nerves, wherein the therapeutic affects neural cells, transporting the therapeutic peripherally through nerves into the CNS and past the blood-brain barrier, and affecting neural cells involved in amyotrophic lateral sclerosis.

### DETAILED DESCRIPTION

Generally, the present invention provides for a method of delivering a treatment to the central nervous system by administering a therapeutic intramuscularly into muscles innervated by cranial nerves and/or spinal nerves, and transporting the therapeutic peripherally through nerves into the central nervous system and to a site of treatment. The present invention further provides for a method of treating a neural disease or a part of the body by administering a compound directly into muscles innervated by cranial or spinal neurons, for example muscles innervated by the motor trigeminal nerve (hereinafter, “MTN”).

The term “neurological disorders” as used herein means disorders associated with the central and peripheral nervous system including, but not limited to, Parkinson's disease, amyotrophic lateral sclerosis (i.e., Lou Gehrig's Disease), other neurodegenerative diseases of the central and peripheral nervous systems, nerve injury or trauma associated with spinal cord injury, and any other neurological disorders known to those of skill in the art.

The phrase “muscles innervated by the MTN” as used herein means muscles including, but not limited to, the masseters, temporalis, lateral and medial pterygoids, other muscles of mastication, and any other muscles innervated by the motor trigeminal nerve known to those of skill in the art.

The term “composition,” “compound,” or “therapeutic” as used herein means a substance that is capable of having an effect on a neural disorder or a part of the body connected to the motor trigeminal nerve. The compounds of the present invention can be used to treat numerous neurological disorders including, but not limited to, Parkinson's disease and nerve damage resulting from spinal cord injuries. The compounds can also be used to treat a part of the body connected to the motor trigeminal nerve.

The term “effective amount” as used herein, means, but is not limited to, the amount determined by such consideration as are known in the art of treating or affecting neurological disorders. The effective amount must be sufficient to provide measurable relief in treated individuals such as exhibiting improvements, nerve growth, nerve regeneration, improved movement, more rapid recovery, regression of symptoms, elimination of symptoms or reduction of complications, or other measurements as appropriate and known to those of skill in the medical arts.

The term “intramuscular” as used herein refers to the intramuscular route (IM route). In the IM route of administration, injections are made into the striated muscle fibers that lie beneath the subcutaneous layer. Volumes injected generally range from 0.5 to 2.0 mL, although higher volumes can be given in certain muscle areas.

The basis of the present invention involves a method of delivery of a therapeutic by the administration of compounds into muscles innervated by specific nerves such as the MTN or other spinal cord nerves in order to transport the compound past the blood-brain barrier and into the CNS. This administration technique is important for those compounds that are incapable of crossing the blood-brain barrier and therefore cannot be given in conventional manners (i.e., oral, intravenous, and/or subcutaneous administration). The mechanism by which the compounds travel in the body is known as axonal transport.

More specifically, axonal transport is the mechanism by which organelles and macromolecules are transported along axons. Molecules can move along tracks made of filamentous organelles (microtubules). Movement of the molecules is accomplished by motor proteins. This movement is essential for the growth and survival of axons and continues throughout the life of the nerve cell. In anterograde axonal transport, molecules are transported along microtubules down the length of the axon to the axon terminals, where they are inserted into the plasma membrane or other organelles. Going in the reverse direction, retrograde axonal transport, damaged membranes and organelles move up the axon towards the cell body in order to be degraded. There are two different components of axonal transport—fast and slow transport. In fast axonal transport, membranous organelles move along microtubules at rates of several hundred millimeters per day, being propelled by motor proteins described below. Fast transport can occur in the anterograde and retrograde directions. Lysosomal vesicles and enzymes in retrograde fast transport can move 200-300 millimeters per day. In slow axonal transport, cytoskeletal and cytosolic proteins move at rates of a few millimeters per day. Slow transport occurs only in the retrograde direction, when materials are degraded. Slow transport is not well understood. Molecular motor proteins are responsible for carrying materials in and out of the axon. There are several large families of these proteins, such as the kinesin, myosin, and dynein families.

There are many motor neuron diseases involving impairment of axonal transport. For example, it is believed that impairment of neurofilaments in slow axonal transport is an underlying factor in amyotrophic lateral sclerosis. Other diseases include hereditary spastic paraplegias and Kennedy's disease. A common characteristic among motor neural diseases is an abnormal protein aggregation in the motor neurons. Another common characteristic is a genetic defect affecting motor proteins.

There are three classes of neurons: sensory neurons, which carry information from the sense organs to the brain; interneurons, which have short axons and communicate only within their immediate region; and motor neurons, which have long axons and carry information from the CNS to muscles and glands in the body. Motor neurons are of interest in the present invention. Because of the extent of the brainstem connectivity, injection of compounds and/or compositions such as GDNF directly into muscles innervated by the MTN can be picked up by the motor neurons and transported retrograde into the MTN where the compounds can be widely distributed to SN as well as other nuclei in the brainstem where a palliative effect would occur. For example, the administration of compounds into the muscles innervated by the MTN is an appropriate avenue of effecting and/or treating areas that are also connected to the MTN. Thus, administration of compounds such as GDNF into these muscles would result in the MTN transporting the compounds into areas where a palliative effect can be exerted. Moreover, using injection of a compound into muscle along with axonal transport as a delivery system is a non-invasive procedure in contrast to the current treatments of direct injection into the brain wherein surgery is required.

There are numerous embodiments of the present invention. One embodiment provides a method of treating neurological disorders such as Parkinson's disease by administering a therapeutic directly into a muscle innervated by the MTN and transporting the therapeutic peripherally through nerves into the CNS and to the site of treatment in order to have an effect on the neural cells associated with Parkinson's disease. Preferably, the muscle is one of the masseters. Preferably, the therapeutic is injected into the muscle. Preferably the therapeutic is GDNF and the amount of GDNF is at least 15 micrograms in each masseter per day or 105 micrograms in each masseter per week. This is a minimum quantity that can be infused directly into each masseter. In practice, the quantity for a masseter injection can be more, since it is an indirect pathway and an unspecified amount can be lost in muscle rather than being taken up in nerve tissue. For example, 45 micrograms can be injected in each masseter per day. In general, the dose is adequate to deliver an effective amount to the site of action.

In another embodiment of the present invention, there is provided a method of treating spinal cord injuries by administering therapeutics such as GDNF into the musculature directly innervated by the spinal cord and transporting the therapeutic peripherally through nerves into the CNS and to the site of treatment in order to have an effect on the neural cells associated with the spinal cord injury. The amount of GDNF administered to the patient is also determined on a patient-by-patient basis.

Another embodiment of the present invention is a method of treating amyotrophic lateral sclerosis by administering an effective amount of a therapeutic, such as a neurotrophic or growth factor that affects neural cells, into musculature innervated by cranial and/or spinal nerves, and transporting the therapeutic peripherally through nerves into the CNS and to the site of treatment. In amyotrophic lateral sclerosis, both the upper motor neurons and the lower motor neurons are diseased. The lower motor neurons are primary motor neurons of the spinal cord and brain stem, which directly innervate the skeletal muscles. The upper motor neurons are neurons that originate higher in the brain and synapse on the lower motor neurons in order to convey descending commands for movement. The therapeutics have an affect on the lower motor neural cells and the upper motor neural cells involved in amyotrophic lateral sclerosis.

In another embodiment, there is provided a method of treating a part of the body that is connected to cranial and or spinal nerves by administering an effective amount of a therapeutic into musculature innervated by cranial and/or spinal nerves, and transporting the therapeutic peripherally through nerves into the CNS and to the site of treatment.

In general, the therapeutic is preferably lipophilic and able to cross cell membranes in order to absorb easily into the patient's muscles. The therapeutic can also be hydrophilic if it is encapsulated or otherwise masked by a lipophilic carrier such as a lysosome or other carrier known in the art. Lysosomes are produced in the Golgi apparatus and contain catabolic enzymes used in the digestion of macromolecules. Lysosomes are common in all cell types of the CNS, and are prominent in neurons. Lysosomes maintain a pH of 5 in their interior allowing the enzymes to function optimally. The membrane surrounding the lysosome has transport proteins to transport molecules from the inside of the lysosome to the outside. Improved lipophilicity also can be attained through a carrier-linked prodrug or emulsions as further described below. Any other suitable method of improving the lipophilicity and improving the transport of the therapeutic can be used.

The therapeutic to be delivered can be selected from many different therapeutics known in the art. For example, the therapeutic can be a neurotrophic factor or growth factor. Neurotrohic factors are proteins that play a role in nerve cell protection and regeneration and which may therefore be useful in treating a variety of neurological disorders. Neurotrophic or growth factors that can be used with the present invention include, but are not limited to, glial-derived neurotrophic factor (hereinafter, “GDNF”), brain-derived neurotrophic factor (hereinafter, “BDNF”), nerve growth factor (hereinafter, “NGF”), and any other similar neurotrophic or growth factors known to those of skill in the art. GDNF is a member of the transforming growth factor-beta superfamily and is the trophic factor for mescephalic dopaminergenic neurons and spinal motoneurons. GDNF has been shown to promote motor axonal growth in the CNS of injured adults.

The therapeutic can be an Alzheimer's therapeutic such as Aricept® (donepezil hydrochloride), Exelon® (rivastigmine tartrate), or Reminyl® (galantamine HBr).

The therapeutic can be an amyotrophic lateral sclerosis therapeutic such as Rilutek® (riluzole).

The therapeutic can be an analgesic such as Excedrin®(D (acetaminophen), Tylenol® (acetaminophen), Ultracet® (tramadol hydrochloride/acetaminophen), Ultram® (tramadol hydrochloride), Carbatrol® (carbamazepine), Lidoderm® (lidocaine), Naropin® (ropivacaine HCl), Neurontin® (gabapentin), Phenergan® (promethazine HCl), Synvisc®) (hyglan G-F 20), Tegretoll® (carbamazepine), Traumeel® Injection Solution, Buprenex® (buprenorphine hydrochloride), Nubain® (nalbuphine hydrochloride), Suboxone® (buprenorphine HCl and naloxone HCl dihydrate), Subutex® (buprenorphine HCl), Actiq® (fentanyl citrate), Astramorph® (morphine sulfate), Avinza® (morphine sulfate), Darvocet® (propoxyphene hydrochloride), Darvon® (propoxyphene napsylate), Demerol® (meperidine hydrochloride), Dilaudid® (hydromorphone hydrochloride), Kadian® (morphine sulfate), Lortab® (hydrocodone bitartrate and acetaminophen), Maxidone® (hydrocodone bitartrate and acetaminophen), MS Contin® (morphine sulfate), MSIR® (morphine sulphate), Norco® (hydrocodone bitartrate and acetaminophen), Numorphan® (oxymorphone hydrochloride), OxyContin® (oxycodone HCl), OxylR® (oxycodone HCl), Percocet® (oxycodone and acetaminophen), Synalgos® (dihydrocodeine bitartrate), Tylenol with Codeine®, Vicodin® (hydrocodone bitartrate and acetaminophen), Zydone® (hydrocodone bitartrate and acetaminophen), Midrin® (isometheptene mucate), Sedapap® (butalbital and acetaminophen), Anaprox® (naproxen sodium), Arthrotec™ (diclofenac sodium and misoprostol), Bextra® (valdecoxib), Cataflame (diclofenac potassium), Celebrex® (celecoxib), Clinoril® (sulindac), Dolobid® (diflunisal), Naprosyn® (naproxen), Feldene® (piroxicam), Indocin® (indomethacin), Mobic® (meloxicam), Motrin® (ibuprofen), Naprelan® (naproxen sodium), Ponstel® (mefenamic acid), Relafen® (nabumetone), Toradol® (ketorolac tromethamine), Trilisate® (choline magnesium trisalicylate), Vioxx® (rofecoxib), Voltaren®) (diclofenac sodium), Excedrin®, Aspirin®, or Pravigard® (buffered aspirin and pravastatin sodium).

The therapeutic can also be an anesthetic such as Brevital® Sodium (methohexital sodium), Diprivan® (propofol), Suprane® (desflurane), Naropin® (ropivacaine HCl), Nesacaine® (chloroprocaine HCl), Polocalne® (mepivacaine hydrochloride), or serapin.

The therapeutic can also be an anticonvulsant such as Mebaral® (mephobarbital), Nembutal® Sodium (pentobarbital sodium), Klonopine (clonazepam), Tranxene® (clorazepate dipotassium), Valium® (diazepam), gabatril (tiagabine hydrochloride), Neurontin® (gabapentin), Cerebyx® (fosphenyloin sodium), Dilantin® (phenyloin), Peganone® (ethotoin), Phenylek® (phenyloin sodium), Carbatrol® (carbamazepine), Depacon® (valproate sodium), Depakene® (valproic acid), Depakote® (divalproex sodium), Felbatol® (felbamate), Keppra® (levetiracetam), Tegretol® (carbamazepine), Topamax® (topiramate), Trileptal® (oxcarbazepine), Zonegran® (zonisamide), Lamictal® (lamotrigine), Celontin® (methsuximide), or Zarontin® (ethosuximide).

The therapeutic can be a narcotic antagonist antidote such as Narcan® (naloxone hydrochloride).

The therapeutic can be an antiparkinsonian agent such as levodopa, carbidopa, perindopril, Cogentin® (Benztropine Mesylate), Nulev® (hyoscyamine sulfate), Comtan® (entacapone), Stalevo® (carbidopa, levodopa, and entacapone), Tasmar® (tolcapone), Mirapex® (pramipexole dihydrochloride tablets), Permax® (pergolide mesylate), Requip® (ropinirole hydrochloride), Symmetrel® (amantadine hydrochloride), and Eldepryl® ((selegiline hydrochloride).

The therapeutic can be a CNS depressant such as Skelaxin® (metaxalone), or a CNS stimulant such as Adderall® (amphetamine), Desoxyn® (methamphetamine hydrochloride), Dexedrine® (dextroamphetamine sulfate), DextroStat® (dextroamphetamine sulfate), Concerta® (methylphenidate HCl), Cylert® (pemoline), Focalin® (dexmethylphenidate hydrochloride), Metadate® (methylphenidate HCl), Provigil® (modafinil), Ritalin® Hydrochloride (methylphenidate HCl), or Strattera® (atomoxetine HCl).

The therapeutic can be an antiemetic gastrointestinal agent such as Aloxi™ (palonosetron hydrochloride), Antivert® (meclizine HCl), Anzemet® (dolasetron mesylate), Compazine® (prochlorperazine), Emend® (aprepitant), Kytril® (granisetron hydrochloride), Marinol® (dronabinol), Phenergan® (promethazine HCl), Tigan® (trimethobenzamide hydrochloride), or Zofran® (ondansetron hydrochloride).

The therapeutic can be a muscle relaxant such as Botox® (botulinum toxin type A), Zemuron® (rocuronium bromide), Dantrium® (dantrolene sodium), Flexeril® (cyclobezaprine HCl), Norflex® (orphenadrine citrate), Soma® (carisoprodol), Valium® (diazepam), or Zanaflex® (tizanidine hydrochloride).

The therapeutic can be an antianxiety agent such as Librium® (chlordiazepoxide HCl), Tranxene® (clorazepate dipotassium), Valium® (diazepam), Xanax® (alprazolam), Atarax® (hydroxyzine hydrochloride), Effexor® (venlafaxine hydrochloride), Paxil® (paroxetine hydrochloride), Sinequan® (doxepin HCl), Vistaril® (hydroxyzine pamoate), or Zoloft® (sertraline hydrochloride).

The therapeutic can be an antidepressant such as Effexor® (venlafaxine hydrochloride), Remeron® (mirtazapine), Wellbutrin® (bupropion hydrochloride), Nardil® (phenelzine sulfate), Parnate® (tranylcypromine sulfate), Celexa® (citalopram hydrobromide), Lexapro® (escitalopram oxalate), Paxil® (paroxetine hydrochloride), Prozac® (fluoxetine hydrochloride), Zoloft® (sertraline hydrochloride), Norpramin® (desipramine hydrochloride), Sinequan® (doxepin HCl), Surmontil® (trimipramine maleate), or Vivactil® (protriptyline HCl).

The therapeutic can be an antimanic agent such as Depakote® (divalproex sodium), Eskalith® (lithium carbonate), or Zyprexa® (olanzapine).

The therapeutic can be an antipanic agent such as Klonopin® (clonazepam), Paxil® (paroxetine hydrochloride), Prozac® (fluoxetine hydrochloride), Xanax® (alprazolam), or Zoloft® (sertraline hydrochloride).

The therapeutic can be an antipsychotic agent such as Abilify® (aripiprazole), Clozaril® (clozapine), Geodon® (ziprasidone), Haldol® (haloperidol), Loxitane® (loxapine succinate), Moban® (molindone hydrochloride), Navane® (thiothixene), Orap® (pimozide), Risperdal® (risperidone), Seroquel® (quetiapine fumarate), thiothixene, Zyprexa® (olanzapine), Compazine® (prochlorperizine), Serentil® (mesoridazine besylate), thioridazine hydrochloride, Sarafem® (fluoxetine hydrochloride), Paxil® (paroxetine hydrochloride), Prozac® (fluoxetine hydrochloride), or Zoloft® (sertraline hydrochloride).

The therapeutic can be any other suitable therapeutic for delivery through the CNS by IM injection. The compound can be a small molecule or a biologic. Furthermore, suitable combinations of drugs can be given. For example, an antidepressant can be given along with an anti-anxiety therapeutic. Preferably, the therapeutic does not deaden the nerve in which it is traveling. If there is a deadening effect, dosing should be adjusted in order to reduce the effect.

The therapeutic of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art. Many of the therapeutics listed above have been well developed pharmacologically and their mechanisms of action are well understood. Therefore, dosing of the above listed therapeutics can be easily modified to be used in an axonal transport environment.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and compound effectiveness. The doses may be single doses or multiple doses over a period of several days, but single doses are preferred.

When administering the compound of the present invention, it can be formulated in a unit dosage injectable form (e.g., solution, suspension, or emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. No. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art. The quantity to be administered will vary for the patient being treated and will vary from about 100 ng/kg of body weight to 100 mg/kg of body weight per day and preferably will be from 100 mg/kg to 10 mg/kg per day.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

1. A method of delivering a treatment to the central nervous system, including the steps of:
administering a therapeutic intramuscularly into muscles innervated by nerves chosen from the group consisting of cranial nerves and spinal nerves; and
transporting the therapeutic peripherally through nerves into the CNS and to the site of treatment.

2. The method according to claim 1, wherein the transporting step is further defined as transporting the therapeutic past the blood-brain barrier.

3. The method according to claim 1, wherein the therapeutic is lipophilic.

4. The method according to claim 1, wherein the therapeutic is hydrophilic and contained in a lipophilic carrier.

5. The method according to claim 1, wherein the therapeutic is chosen from the group consisting of neurotrophic factors or growth factors.

6. The method according to claim 5, wherein the neurotrophic factor is chosen from the group consisting of GDNF or BDNF.

7. The method according to claim 5, wherein the growth factor is NGF.

8. The method according to claim 1, wherein the therapeutic is an Alzheimer's therapeutic.

9. The method according to claim 1, wherein the therapeutic is an amyotrophic lateral sclerosis therapeutic.

10. The method according to claim 1, wherein the therapeutic is an analgesic.

11. The method according to claim 1, wherein the therapeutic is an anesthetic.

12. The method according to claim 1, wherein the therapeutic is an anticonvulsant.

13. The method according to claim 1, wherein the therapeutic is a narcotic antagonist antidote.

14. The method according to claim 1, wherein the therapeutic is an anti-Parkinsonian agent.

15. The method according to claim 1, wherein the therapeutic is chosen from the group consisting of CNS depressants or CNS stimulants.

16. The method according to claim 1, wherein the therapeutic is an anti-emetic gastrointestinal agent.

17. The method according to claim 1, wherein the therapeutic is a muscle relaxant.

18. The method according to claim 1, wherein the therapeutic is an anti-anxiety agent.

19. The method according to claim 1, wherein the therapeutic is an antidepressant.

20. The method according to claim 1, wherein the therapeutic is an anti-manic agent.

21. The method according to claim 1, wherein the therapeutic is an anti-panic agent.

22. The method according to claim 1, wherein the therapeutic is an anti-psychotic agent.

23. The method according to claim 1, wherein the therapeutic is a combination of therapeutics chosen from the group consisting of the therapeutics of claims 5, 8, 9, 10, 11, 12, 13, 14, 15, 16, 17, 18, 19, 20, 21, or 22.

24. A method of treating a neural disorder, including the steps of:
administering intramuscularly an effective amount of a therapeutic into muscles innervated by nerves chosen from the group consisting of cranial nerves and spinal nerves;
transporting the therapeutic peripherally through nerves into the CNS; and
affecting neural cells.

25. The method of claim 24, wherein the transporting step is further defined as transporting the therapeutic past the blood-brain barrier.

26. The method according to claim 24, wherein the therapeutic is lipophilic.

27. The method according to claim 24, wherein the therapeutic is hydrophilic and contained in a lipophilic carrier.

28. The method according to claim 24, wherein the therapeutic is chosen from the group consisting of neurotrophic factors or growth factors.

29. The method according to claim 28, wherein the neurotrophic factor is chosen from the group consisting of GDNF or BDNF.

30. The method according to claim 28, wherein the growth factor is NGF.

31. The method according to claim 24, wherein the therapeutic is an Alzheimer's therapeutic.

32. The method according to claim 24, wherein the therapeutic is an amyotrophic lateral sclerosis therapeutic.

33. The method according to claim 24, wherein the therapeutic is an analgesic.

34. The method according to claim 24, wherein the therapeutic is an anesthetic.

35. The method according to claim 24, wherein the therapeutic is an anticonvulsant.

36. The method according to claim 24, wherein the therapeutic is a narcotic antagonist antidote.

37. The method according to claim 24, wherein the therapeutic is an anti-Parkinsonian agent.

38. The method according to claim 24, wherein the therapeutic is chosen from the group consisting of CNS depressants or CNS stimulants.

39. The method according to claim 24, wherein the therapeutic is an anti-emetic gastrointestinal agent.

40. The method according to claim 24, wherein the therapeutic is a muscle relaxant.

41. The method according to claim 24, wherein the therapeutic is an anti-anxiety agent.

42. The method according to claim 24, wherein the therapeutic is an antidepressant.

43. The method according to claim 24, wherein the therapeutic is an anti-manic agent.

44. The method according to claim 24, wherein the therapeutic is an anti-panic agent.

45. The method according to claim 24, wherein the therapeutic is an anti-psychotic agent.

46. The method according to claim 24, wherein the therapeutic is a combination of therapeutics chosen from the group consisting of the therapeutics of claims 28, 31, 32, 33, 34, 35, 36, 37, 38, 39, 40, 41, 42, 43, 44, or 45.

47. A method of treating a part of the body, including the steps of:
administering intramuscularly an effective amount of a therapeutic into muscles innervated by nerves chosen from the group consisting of cranial nerves and spinal nerves;
transporting the therapeutic peripherally through nerves into the CNS; and
affecting neural cells.

48. The method of claim 47, wherein the transporting step is further defined as transporting the therapeutic across the blood-brain barrier.

49. The method according to claim 47, wherein the therapeutic is lipophilic.

50. The method according to claim 47, wherein the therapeutic is hydrophilic and contained in a lipophilic carrier.

51. The method according to claim 47, wherein the therapeutic is chosen from the group consisting of neurotrophic factors or growth factors.

52. The method according to claim 51, wherein the neurotrophic factor is chosen from the group consisting of GDNF or BDNF.

53. The method according to claim 51, wherein the growth factor is NGF.

54. The method according to claim 47, wherein the therapeutic is an Alzheimer's therapeutic.

55. The method according to claim 47, wherein the therapeutic is an amyotrophic lateral sclerosis therapeutic.

56. The method according to claim 47, wherein the therapeutic is an analgesic.

57. The method according to claim 47, wherein the therapeutic is an anesthetic.

58. The method according to claim 47, wherein the therapeutic is an anticonvulsant.

59. The method according to claim 47, wherein the therapeutic is a narcotic antagonist antidote.

60. The method according to claim 47, wherein the therapeutic is an anti-Parkinsonian agent.

61. The method according to claim 47, wherein the therapeutic is chosen from the group consisting of CNS depressants or CNS stimulants.

62. The method according to claim 47, wherein the therapeutic is an anti-emetic gastrointestinal agent.

63. The method according to claim 47, wherein the therapeutic is a muscle relaxant.

64. The method according to claim 47, wherein the therapeutic is an anti-anxiety agent.

65. The method according to claim 47, wherein the therapeutic is an antidepressant.

66. The method according to claim 47, wherein the therapeutic is an anti-manic agent.

67. The method according to claim 47, wherein the therapeutic is an anti-panic agent.

68. The method according to claim 47, wherein the therapeutic is an anti-psychotic agent.

69. The method according to claim 47, wherein the therapeutic is a combination of therapeutics chosen from the group consisting of the therapeutics of claims 51, 54, 55, 56, 57, 58, 59, 60, 61, 62, 63, 64, 65, 66, 67, or 68.

70. A method of treating a spinal cord injury, including the steps of
administering an effective amount of a therapeutic into musculature directly innervated by the spinal cord;
transporting the therapeutic peripherally through nerves into the CNS and past the blood-brain barrier; and
affecting neural cells.

71. The method of claim 70, wherein the compound is GDNF.

72. A method of treating Parkinson's disease, including the steps of
administering an effective amount of a therapeutic into musculature innervated by the motor trigeminal nerve, said therapeutic affecting neural cells;
transporting the therapeutic peripherally through nerves into the CNS and past the blood-brain barrier; and
affecting neural cells involved in Parkinson's disease.

73. The method of claim 72, wherein the compound is GDNF.

74. The method of claim 73, wherein the musculature is at least one masseter muscle.

75. The method of claim 74, further defining said administering step as injecting a pharmaceutical formulation comprising at least 15 micrograms of GDNF once a day in a masseter muscle.

76. The method of claim 74, further defining said administering step as injecting at least 105 micrograms of GDNF in a pharmaceutical formulation per week in a masseter muscle.

77. A method of treating amyotrophic lateral sclerosis, including the steps of
administering an effective amount of a therapeutic into musculature innervated by nerves chosen from the group consisting of cranial and spinal nerves, said therapeutic affecting neural cells;
transporting the therapeutic peripherally through nerves into the CNS and past the blood-brain barrier; and
affecting neural cells involved in amyotrophic lateral sclerosis.
